(19) United States
(12) Patent Application Publication
(10) Pub. No.: US 2013/0304118 A1
(43) Pub. Date: Nov. 14, 2013

(54) Devices and Methods for Vascular Closure

(71) Applicant: University of Rochester, Rochester, NY (US)

(72) Inventors: Rachel Rakvica, Fairport, NY (US); Amy Lerner, Rochester, NY (US); Ankur Chandra, Rochester, NY (US)

(73) Assignee: University of Rochester, Rochester, NY (US)

(21) Appl. No.: 13/893,283

(22) Filed: May 13, 2013

Related U.S. Application Data
(60) Provisional application No. 61/646,305, filed on May 13, 2012.

(51) Int. Cl. A61B 17/00

(52) U.S. Cl. CPC A61B 17/0057
USPC 606/213

(57) ABSTRACT

Devices and methods for vascular closure are disclosed. The invention may be an apparatus having a first catheter comprising a first balloon lumen and a support balloon in pneumatic communication with the first balloon lumen, a sheath having a hood portion, a second catheter comprising a second balloon lumen and an expander balloon in pneumatic communication with the second balloon lumen, and a pusher assembly having a bandage. A method of using such an apparatus may comprise the steps of inserting the apparatus into the vasculature through the puncture, positioning the sheath outside of the puncture, inflating the support balloon, inserting the second catheter into the sheath, inflating the expander to expand the hood portion of the sheath, deflating the expander balloon, positioning the pusher assembly within the hood portion, deflating the support balloon, removing the first catheter, and affixing the bandage on the puncture.

### CROSS-REFERENCE TO RELATED APPLICATIONS

This application claims priority to U.S. Provisional Application No. 61/646,305, filed on May 13, 2012, now pending, the disclosure of which is incorporated herein by reference.

### FIELD OF THE INVENTION

The invention relates to minimally invasive surgical techniques, and in particular to apparatuses and methods for vascular closure.

### BACKGROUND OF THE INVENTION

There are more than 12 million endovascular, minimally invasive-interventional, surgeries performed ever year, with the estimated market value for vascular closure devices at $2 billion. These statistics do not include new procedures and devices such as, percutaneous aortic valves that reduce or eliminate the need for invasive open-heart surgery. However, since these valves and other devices (e.g., aortic endografts used to prevent aortic aneurysms) require the use of sheath sizes that are 18 to 24 Fr., a cardiologist is not able to deliver the device without the presence of a surgeon during the procedure.

Currently, cardiologists are not able to do a procedure in which the sheath size is greater than 10 Fr. because manual compression cannot be used. In such procedures, a surgeon must be present to perform a cut-down through the tissue to expose the vessel and stitch the vessel closed. Stitching the vessel closed is time consuming, taking anywhere from 15-30 minutes, requires the surgeon to make a 3-5″ long incision. Such incisions cause the patient pain, and bring risks such as infection. In cases where manual compression can be used, if the patient is anti-coagulated, a nurse must wait before applying manual compression because the patient is unable to form clots.

Currently available closure devices only close holes from sheaths ranging from 5 to 8 Fr., and are not recommended for patients with vessels that are significantly calcified. Accordingly, there is a need for a vascular closure apparatus and method capable of closing holes caused by sheaths of any size, and especially large sizes (e.g., greater than 10 Fr.).

### BRIEF SUMMARY OF THE INVENTION

The invention may be described as an apparatus for closing a puncture within a vasculature. The apparatus comprises a first catheter. The first catheter has a proximal end, a distal end, a first balloon lumen, and a support balloon. The support balloon is positioned at the distal end of the first catheter and is in pneumatic communication with the first balloon lumen. 3. The support balloon may be configured to be inflated and/or deflated in situ by way of the first balloon lumen. In another embodiment, the support balloon is made from a compliant material capable of providing support for the vasculature without deformation.

In one embodiment, a guide wire may be located in the vasculature. In this embodiment, the first catheter may have a guide lumen configured such that the first catheter is advanced along the guide wire through the puncture into the vasculature.

The apparatus further comprises a sheath having a distal end and a hood portion. The hood portion positioned at the distal end of the sheath. The sheath may be configured to be coaxial with, and in radially-spaced-apart relation to the first catheter. In one embodiment, the sheath may be made from poly(urethane). In another embodiment, the hood portion is configured to expand from a first diameter to a second diameter. The second diameter may be larger than the diameter of the puncture and also larger than the diameter of the sheath. The second diameter may be large enough to form an operable space over the puncture. In one embodiment, the hood portion is made from a shape memory material.

In one embodiment, the first catheter may also have a bleed-back lumen which terminates at a bleed-back orifice. The bleed-back orifice may be positioned on the first catheter proximal to the support balloon. In one embodiment, substantially all of the hood portion of the sheath is located proximal to the bleed-back orifice.

The apparatus further comprises a second catheter having a distal end, a second balloon lumen, and an expander balloon. The expander balloon is positioned at the distal end of the second catheter and is in pneumatic communication with the second balloon lumen. In one embodiment, the expander balloon is configured to be inflated and/or deflated in situ by way of the second balloon lumen to expand the hood portion of the sheath. In one embodiment, the second catheter is configured to be coaxial with, and located radially between, the first catheter and the sheath.

The apparatus further comprises a pusher assembly having a distal end, a pusher, and a bandage. The pusher assembly is configured to be coaxial with, and located radially between, the first catheter and the sheath. The pusher is positioned at the distal end of the pusher assembly. The bandage may be positioned at the distal end of the pusher and may be configured to held in place on the puncture by the pusher. In one embodiment, the bandage has an orifice. In this embodiment, the first catheter may be positioned through the orifice such that the distal end of the first catheter remains inside the vasculature when the bandage is being held on the puncture by the pusher.

In one embodiment, the pusher has an expanded configuration and a collapsed configuration for insertion through an accessory lumen. In another embodiment the pusher is configured to be biased to the expanded configuration such that when the pusher assembly is advanced through the sheath and reaches the hood portion, the pusher will expand to the expanded configuration. In one embodiment, the pusher comprises a plurality of push rods substantially parallel to one another when the pusher is in the collapsed configuration. The pushrods may be configured to expand outwardly with respect to a central, longitudinal axis of the pusher.

In one embodiment, the apparatus may further comprise a lock for locking each component of the apparatus to each other to allow the proper longitudinal position of each component during use.

The invention may also be described as a method for closing a puncture in a vasculature. The method may use an apparatus having a first catheter comprising a first balloon lumen and a support balloon in pneumatic communication with the first balloon lumen, a sheath having a hood portion, a second catheter comprising a second balloon lumen and an expander balloon in pneumatic communication with the second balloon lumen, and a pusher assembly having a bandage.

The method may comprise the steps of inserting the first catheter into the vasculature through the puncture and positioning the sheath outside of the puncture.

The method further comprises the step of inflating the support balloon using the first balloon lumen. The support balloon may be inflated until the balloon is substantially the same diameter as the vasculature.

The method further comprises the step of inserting the second catheter into the sheath. The second catheter is inserted such that the expander balloon is positioned in the hood portion of the sheath.

The method further comprises the steps of inflating the expander balloon using the second balloon lumen to expand the hood portion of the sheath and deflating the expander balloon using the second balloon lumen.

The method further comprises the steps of positioning the pusher assembly within the hood portion of the sheath and deflating the support balloon using the first balloon lumen.

The method further comprises the steps of removing the first catheter from the vasculature and using the pusher assembly to affix the bandage on the vascular puncture to close the puncture.

In one embodiment, the first catheter further comprises a bleed-back orifice and a bleed-back indicator. The bleed-back orifice may be positioned on the first catheter proximal to the support balloon. In such an embodiment, the method may further comprise the steps of advancing the first catheter within the vasculature until the bleed-back indicator shows that the bleed-back orifice is within the vasculature and withdrawing the apparatus until the bleed-back indicator shows that the bleed-back orifice is no longer within the vasculature.

The invention may also be described as a method for minimally-invasive closure of a puncture in a blood vessel. In one embodiment, the method comprises the steps of disposing a support balloon in the blood vessel at the location of the puncture, inflating the support balloon, deploying a hood over the puncture, pushing a bandage onto the blood vessel at the puncture and within the hood, and removing the support balloon and hood.

### DESCRIPTION OF THE DRAWINGS

For a fuller understanding of the nature and objects of the invention, reference should be made to the following detailed description taken in conjunction with the accompanying drawings, in which:

FIG. 1 is a partial diagram of a procedure sheath and guide wire in place in a vessel following a procedure;

FIG. 2 is a partial diagram of a first catheter and a deployment sheath according to an embodiment of the present invention and a guide wire;

FIG. 3 is a partial diagram of the device of FIG. 2, wherein the assembly is withdrawn to a functional position;

FIG. 4 is a partial diagram of the device of FIGS. 2 and 3, wherein the support balloon has been inflated;

FIG. 5 is a partial diagram of the device of FIGS. 2-4, wherein the hooded portion is expanded;

FIG. 6 is a partial diagram of the device of FIGS. 2-5, showing internal detail;

FIG. 7 is a partial diagram of the device of FIGS. 2-6, wherein the expander balloon has been deflated;

FIG. 8 is a partial diagram of the device of FIGS. 2-7, wherein the pusher assembly is in the deployment sheath in a collapsed configuration;

FIG. 9 is a partial diagram of the device of FIGS. 2-8, wherein the pusher assembly has advanced and the pusher and bandage have expanded in the hooded portion;

FIG. 10 is a partial diagram of the device of FIGS. 2-9, wherein the bandage is on the vessel wall;

FIG. 11 is a partial diagram of the device of FIGS. 2-10, wherein the first catheter has been withdrawn;

FIG. 12 depicts the bandage in place;

FIG. 13 depicts one embodiment of a bandage with an orifice;

FIG. 14 is a diagram of a first catheter and a deployment sheath of a device according to an embodiment of the present invention;

FIG. 15 depicts a valve of a catheter used as an exemplary lock of the present invention;

FIG. 16 is a flowchart showing a method for closing a puncture in a vasculature according to the present invention; and

FIG. 17 is a flowchart showing a method for minimally-invasive closure of a puncture in a blood vessel according to the present invention.

### DETAILED DESCRIPTION OF THE INVENTION

The present invention describes vascular closure apparatuses and methods in which holes greater than 10 Fr., even in diseased vessels, can be closed percutaneously, without a cut-down, and potentially without the need for a surgeon.

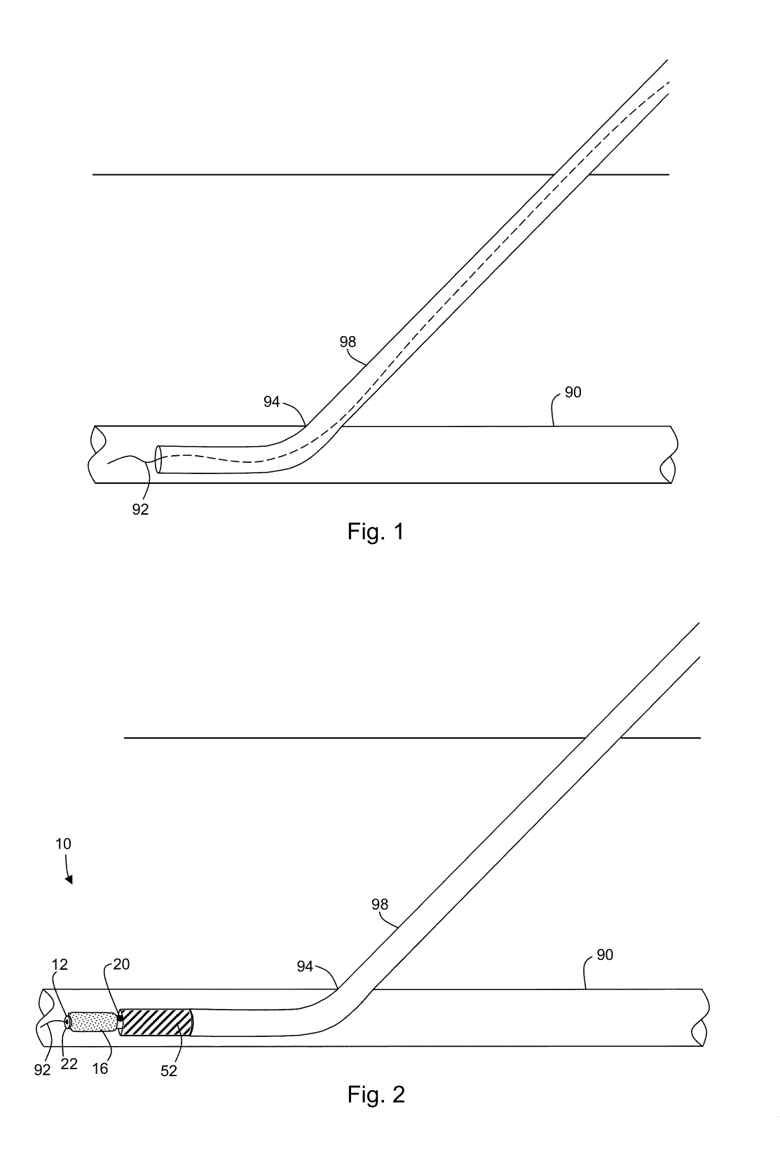

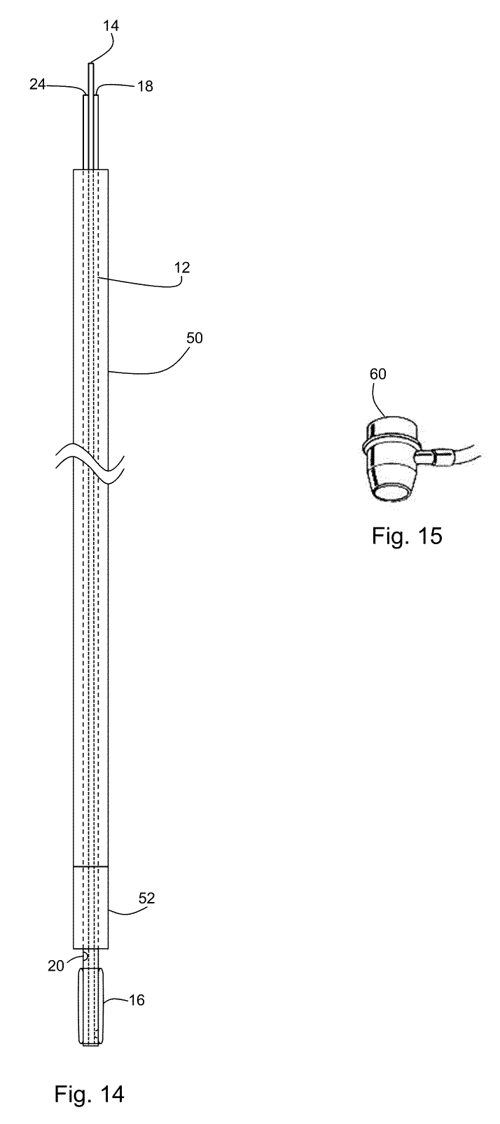

The present invention may be embodied as an apparatus 10 for closing a vascular puncture 94. FIGS. 2 and 14 illustrate one such embodiment. The apparatus 10 comprises a first catheter 12 having a proximal end and a distal end 22. The first catheter 12 includes a guide lumen 14. The guide lumen 14 is configured such that the first catheter 12 may be advanced into an individual along a guide wire 92 already located in the individual. FIG. 1 illustrates one such embodiment having a guide wire 92. The first catheter 12 further comprises a support balloon 16 at the distal end 22. The support balloon 16 is in pneumatic communication with a balloon lumen 18, and the support balloon 16 is configured to be inflated and/or deflated in situ by way of the balloon lumen 18. FIG. 4 illustrates an inflated support balloon 16. The support balloon 16 may be made from a compliant balloon material, for example, able to provide support for the vessel walls without causing deformation. The first catheter 12 comprises a bleed-back lumen 24 which terminates at a bleed-back orifice 20. The bleed-back orifice (bleed-back valve) 20 is located on the first catheter 12 at a position proximal from the support balloon 16. The bleed-back orifice 20 is configured to allow the operator of the apparatus 10 to determine if the first catheter 12 is positioned in the individual such that the bleed-back orifice 20 is in a blood vessel 90 or not in a blood vessel 90.

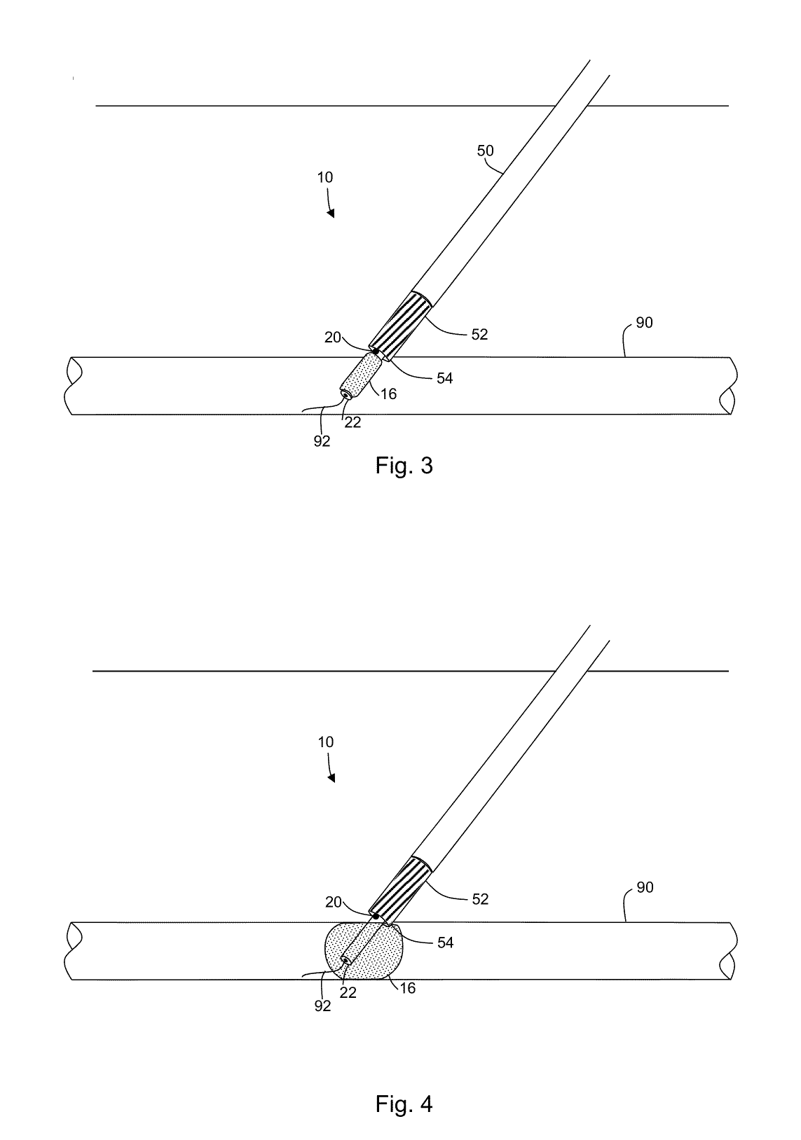

The apparatus 10 further comprises a deployment sheath 50 having a hood portion 52 at a distal end 54 of the deployment sheath 50. These elements are shown, for example, in FIG. 3. The deployment sheath 50 is configured to be coaxial with and in radially spaced apart relation with the first catheter 12 such that other coaxial devices may be advanced or removed between the first catheter 12 and the deployment sheath 50. The deployment sheath 50 may be made from any suitable material, such as, for example, poly(urethane). The distal end 54 of the deployment sheath 50 is configured such that substantially all of the hood portion 52 is located at a proximal position with respect to the bleed-back orifice 20 of the first catheter 12. The hood portion 52 is configured to be expandable in situ. As such, the hood portion 52 is made from a material and/or structured to move from a collapsed configuration (wherein the hood portion 52 is collapsed to a diameter which is smaller than, the same as, or slightly larger than the diameter of the deployment sheath 50) to an expanded configuration (wherein the diameter of the hood portion 52 is larger than the diameter of the deployment sheath 50 and forms an operable space). In an exemplary embodiment, the hooded portion 52 may be made from a shape memory material, for example, a shape memory polymer (“SMP”). In another embodiment, the hooded portion 52 is made from nitinol. Other materials will be apparent in light of the current disclosure. The hooded portion 52 may be integral to the deployment sheath 50 or may be a separate component affixed to the deployment sheath 52. Other configurations will be apparent in light of this disclosure. The diameter of the hood portion 52 is preferably larger than the vascular puncture which requires closure. In this way, a bandage installed by the apparatus 10 has an area larger than the vascular puncture 94—i.e., having a margin around the vascular puncture 94.

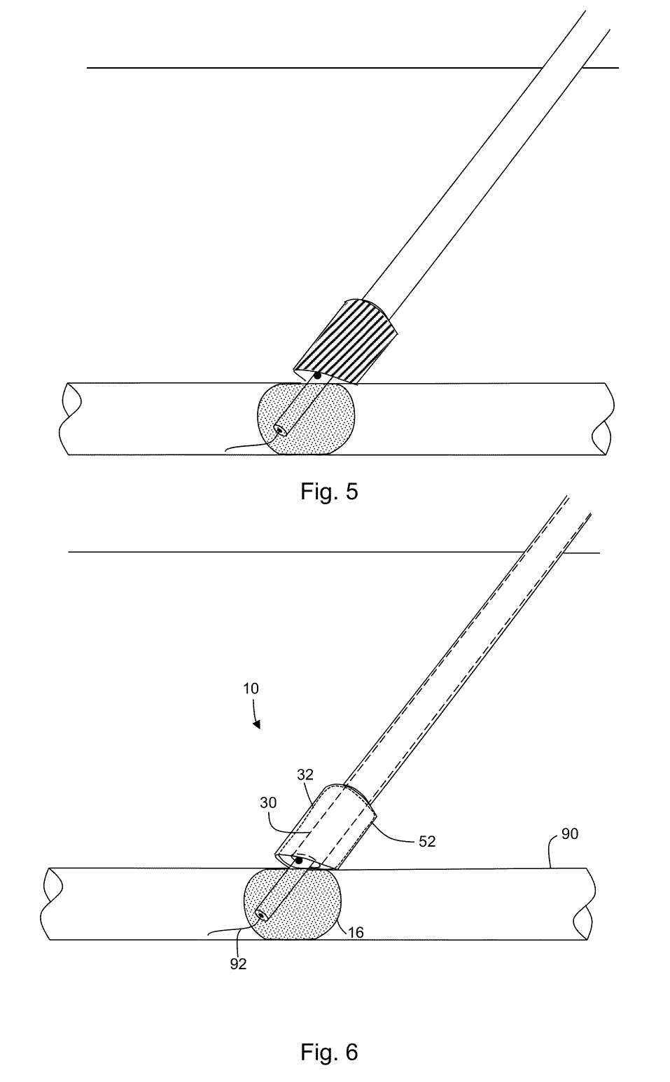

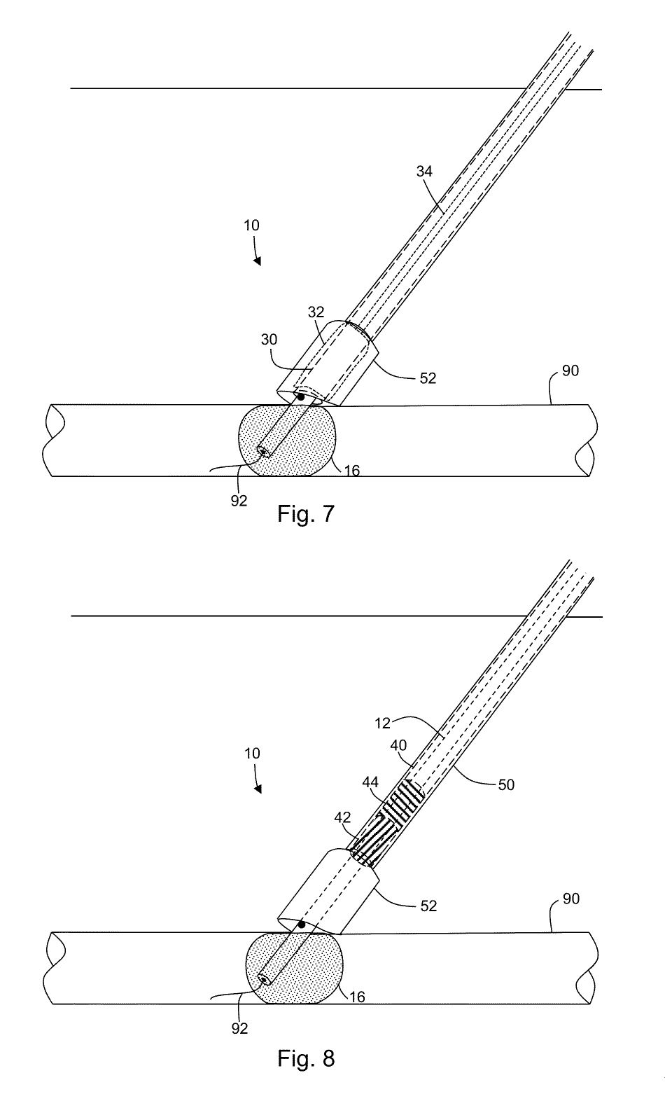

The apparatus 10 further comprises a second catheter 30 comprising an expander balloon 32 at a distal end. FIGS. 5, 6, and 7 illustrate one embodiment of the expander balloon 32 in use. The second catheter 30 has a expander balloon lumen 34 in pneumatic communication with the expander balloon 32 such that the expander balloon 32 may be inflated and/or deflated by an operator of the apparatus 10. The second catheter 30 is configured to be coaxial with the first catheter 12 and the deployment sheath 50 and located (radially) between the first catheter 12 and the deployment sheath 50. Once the second catheter 30 is advanced such that the expander balloon 32 reaches the distal end 22 of the deployment sheath 50 (i.e., the expander balloon 32 is substantially at a same longitudinal position as the hood portion 52), the expander balloon 32 may be inflated to expand the hood portion 52 of the deployment sheath 50. As such, the expander balloon 32 may be made from a stiff balloon material. The expander balloon 32 may then be deflated and the second catheter 30 removed from the deployment sheath 50. The hood portion 28 will generally remain expanded. The second catheter 30 may be embodied with other known expanders in place of the expander balloon 32. Any device which can be selectively expanded and contracted in situ to reconfigure the hood portion 52 may be used.

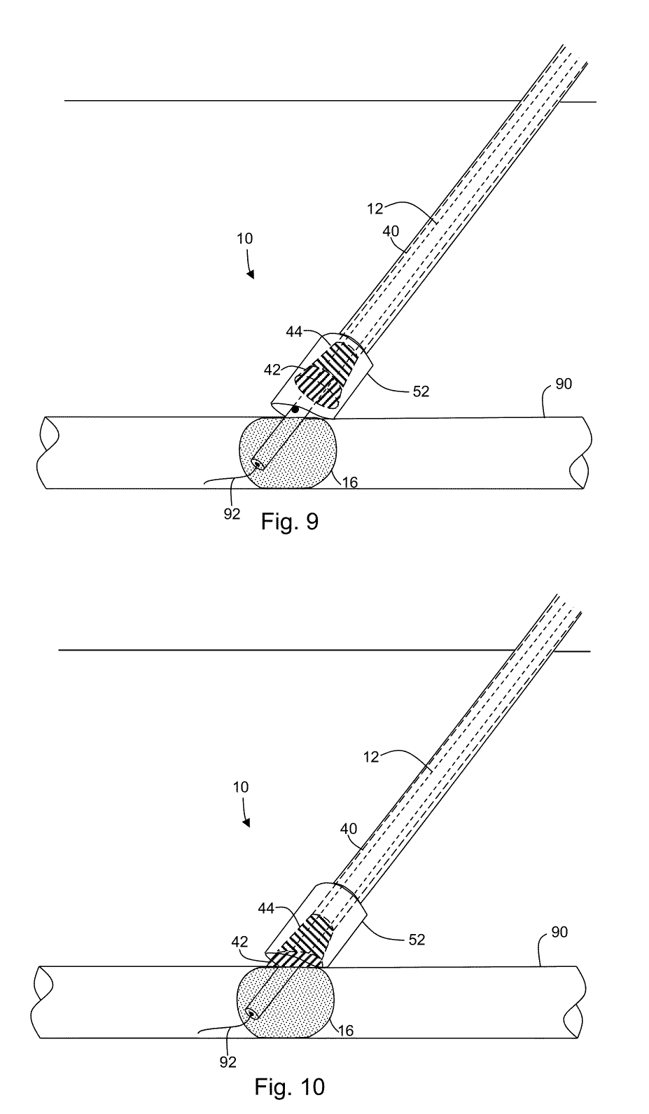

The apparatus 10 further comprises a pusher assembly 40. One example of the pusher assembly 40 can be seen in FIGS. 8, 9, and 10. The pusher assembly 40 is configured to be coaxial with the first catheter 12 and the deployment sheath 50 and located (radially) between the first catheter 12 and the deployment sheath 50. A distal end of the pusher assembly 40 has a pusher 44, which has a collapsed configuration and an expanded configuration. The pusher 44 may be configured to be biased to the expanded configuration such that when the pusher assembly 40 is advanced through the deployment sheath 50 and reaches the hooded portion 52, the pusher 44 will expand to its expanded configuration within the operable space of the hooded portion 52. In one embodiment, the pusher 44 comprises a plurality of push rods, which are substantially parallel to one another when the pusher 44 is in the collapsed state. In this embodiment, the push rods are biased to an expanded position wherein a distal end of the push rods will be urged outwardly (away from a central, longitudinal axis of the plurality of push rods). As such, when the pusher assembly 40 is retrieved from the deployment sheath 12, the push rods will move back into the collapsed configuration and the pusher assembly 40 may once again move between the first catheter 12 and the deployment sheath 52. The pusher assembly 40 may comprise an actuator, such as, for example, a lever, for control of the pusher 44 by the operator.

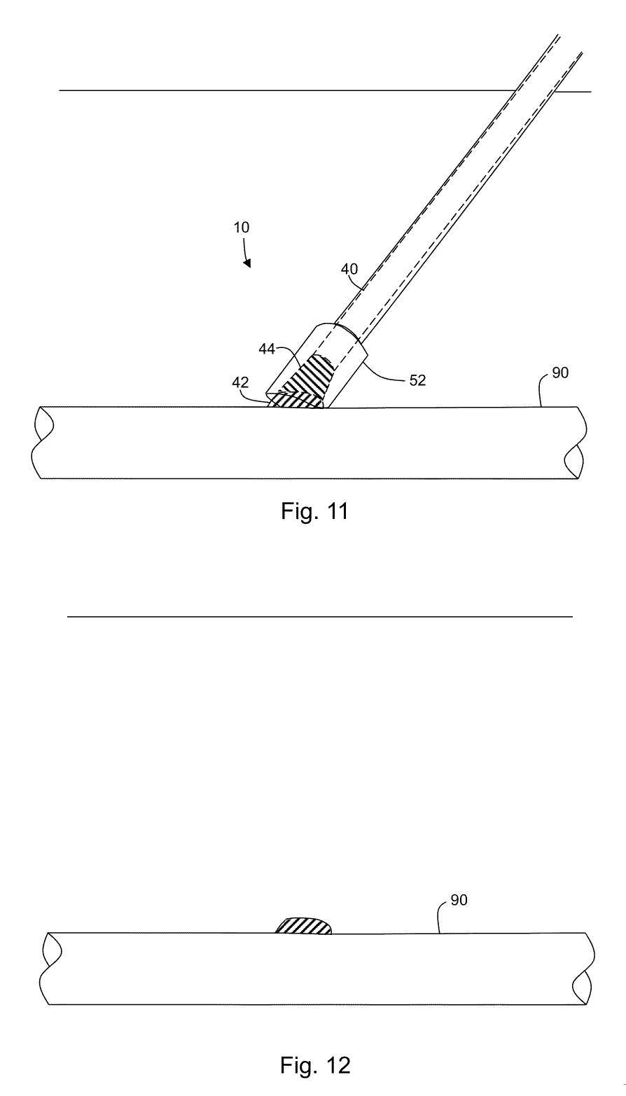

A bandage 42 is positioned at the distal end of the pusher 44. FIGS. 11 and 12 illustrate the bandage 42 being placed over the puncture 94. The bandage 42 is made from a biocompatible material suitable for closure of the vascular puncture 94. The bandage 42 may be made from, for example, a swellable/expandable hydrogel, such as poly(ethylene glycol); bioabsorbable materials; biodegradable materials; polyester; polyurethane; silicone; polytetrafluoroethylene (PTFE); and combinations/composites of these and/or other materials. The bandage 42 is configured to be moved to the operable space of the hood portion 52 and pushed and held in position over the vascular puncture 94 by the operator's use of the pusher assembly 40. It should be noted that the bandage 42 will comprise an orifice 43 through which the first catheter 12 will be disposed such that that the distal end 22 of the first catheter 12 can remain inside the vessel 90 when the bandage 42 is in place on the vessel 90 over the puncture 94 to be repaired.

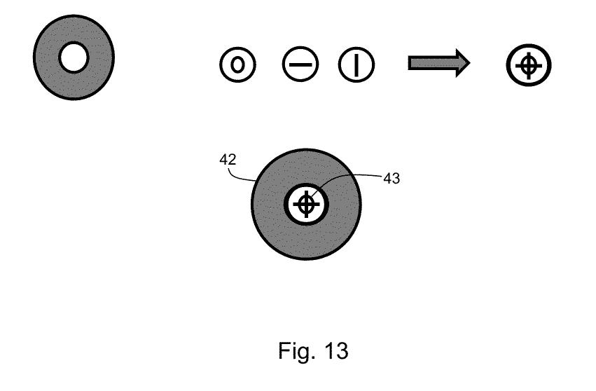

The orifice 43 of the bandage 42 may be configured in the manner pictured in FIG. 13 or other configurations urging a sealing relation in the orifice 43. The orifice 43 may be configured as a no-bleed back of a catheter valve. In this way, when the distal end 22 of the first catheter 12 is retracted from the vessel 90, the orifice 43 be urged to seal itself. The bandage 42 may further comprise a hemostatic agent to assist with sealing upon removal of the first catheter 12. The bandage 42 may further comprise a fixing agent, such as an adhesive, to better cause the bandage 42 to remain in place once the apparatus 10 is removed. The fixing agent may be on a side of the bandage 42 touching the vessel 90 and/or the fixing agent may be on the side opposite the vessel 90 such as a self-expanding hydrogel to hold the bandage 42 in place by pressure.

The apparatus 10 may further comprise a lock 60, as shown in FIG. 15, for locking each component of the apparatus 10 (e.g., any combination of the first catheter 12, the deployment sheath 50, the second catheter 30, and pusher assembly 40) to each other. In this manner, an operator using the apparatus 10 will be assured of the proper longitudinal position of each component during use.

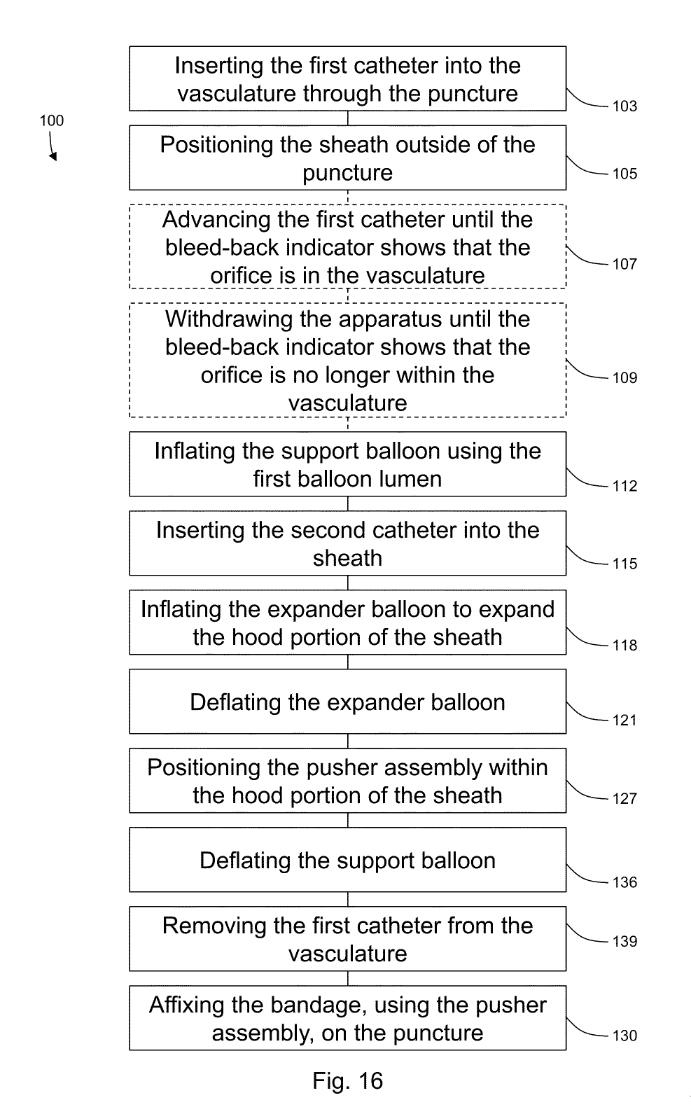

The present invention may be embodied as a method 100 for vascular closure using the apparatus 10 described above (or other embodiments of the apparatus). FIG. 16 is a flowchart showing embodiments of the method 100. During a surgical procedure, or minimally invasive surgery, a vascular puncture will be created. Upon completion of the surgery, a guide wire 92 will remain in position with a proximal end in an extra corporeal location, and a distal end in the blood vessel. A procedure sheath 98 may also be in place. The method comprises the step of inserting 103 the first catheter 12 into the individual over the guide wire 92. Where the procedure sheath 98 is in place, the first catheter 12 is inserted through the procedure sheath 98. The deployment sheath 50 may preferably be inserted with the first catheter 12, or the first catheter 12 and the deployment sheath 50 may be inserted separately. The first catheter 12 is advanced 107 until the bleed-back indicator shows that the bleed-back valve 20 is within the blood vessel 90. As such, the operator will know the distal end 22 of the first catheter 12 has arrived at the site and is within the vessel 90. The components may be locked 106 to one another and to the procedure sheath 98 using, for example, the lock 60. For convenience, the components in place at any given time are referred to as the “assembly.”

The method 100 further comprises the step of withdrawing 109 the assembly until no blood is indicated in the bleed-back indicator (i.e., the bleed-back valve 20 is no longer within the vessel 90). In this way, the operator will know that the support balloon 16 of the first catheter 12 is within the vessel 90 and the elements of the apparatus 10 which are on a proximal side of the balloon are located outside of the vessel 90.

The method 100 comprises the step of inflating 112 the support balloon 16. The support balloon 16 is inflated 112 until the balloon is substantially the same diameter as the vessel (the diameter of each as measured with reference to a cross-section of the vessel). The second catheter 30 is inserted 115 into the deployment sheath 50 until the expander balloon 32 is in position under the hood portion 52. The second catheter 30 may be inserted 115 at the same time as the first catheter 12 and/or the deployment sheath 50, or the second catheter 30 may be inserted 115 independently.

The expander balloon 32 is inflated 118 to, in turn, expand the hooded portion 52 of the deployment sheath 50. The expander balloon 32 may be inflated 118 to a pre-determined pressure. The expander balloon 32 is deflated 121 and the second catheter 30 is withdrawn 124 from the individual.

The method 100 comprises the step of inserting 127 the pusher assembly 40 into the individual over the first catheter 12 and within the deployment sheath 50. The pusher assembly 40 may be inserted 127 until it is locked into position on the deployment sheath 50, indicating that it is position for installation of the bandage 42. The operator will affix 130 the bandage 42 into position using the actuator 48. Affixing 130 the bandage 42 may further comprise the step of holding 133 the bandage 42 in position for a pre-determined length of time, such as, for example, one minute.

The support balloon 16 is deflated 136 and the first catheter 12 is removed 139. The bandage 42 may be held in place for a time period after removal of the first catheter 12 to allow the orifice 43 to be better sealed through, for example, clotting. The assembly is removed 142, leaving the bandage 42 in place on the vessel 90. The expanded hooded portion 52 will generally fit through the individual's tissues without problem. Where adjustment needs to be made to allow passage of the expanded hooded portion 52, for example, at the individual's skin, the tissue may be cut in order to allow passage of the assembly.

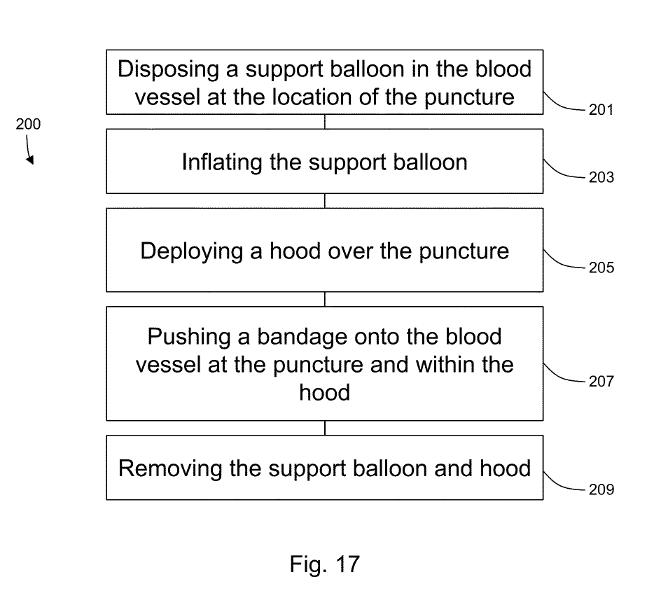

The present invention may also be embodied as a method 200 for minimally-invasive closure of a puncture in a blood vessel as shown in FIG. 17. The method 200 comprises disposing 201 a support balloon in the blood vessel at the location of the puncture. The method 200 further comprises the step of inflating 203 the support balloon. The method 200 further comprises the step of deploying 205 a hood over the puncture. The method 200 further comprises pushing 207 a bandage onto the blood vessel at the puncture and within the hood. The method further comprises removing 209 the support balloon and hood.

Exemplary Calculated Measurements

The following analysis is a non-limiting example of determining the force needed to stop the bleeding directly at the site of closure on hole created by 12 or 18 Fr. sheath:

Assumptions:

Hole created is a circle and is equal to area of the sheath. For example, there is no elastic recoil of the vessel to partially close the hole. (Normally, the vessel would have some elastic recoil and the hole would be more in the shape of a slit or half-moon, assuming a circle overestimates the force—adding a safety margin).

Systolic blood pressure is between 120 mmHg and 180 mmHg (180 mmHg was chosen as the upper bound because closure devices are not recommended for patients with a systolic blood pressure >180 mmHg).

$$r = \frac{Fr}{6}$$

$$area = π {r}^{2}$$

$$F = P * A$$

Example calculation for 12 Fr. hole at 120 mmHg:

$$F = ( 120 mmHg ) ( \frac{133.322 \frac{N}{{m}^{2}}}{1 mmHg} ) ( 4 π {mm}^{2} ) {( \frac{1 m}{1000 mm} )}^{2}$$

$$F = 0.201 N$$

| | 12 Fr. hole | 18 Fr. hole |
|---|---|---|
| Radius | 2 mm | 3 mm |
| Area | 4π mm² | 9π mm² |
| Pressure | 120 mmHg:180 mmHg | 120 mmHg:180 mmHg |
| (Systolic Pressure) | | |
| Force (Newtons) | 0.201:0.302 | 0.452:0.679 |
| Stress (MPa) | 0.016:0.024 | 0.016:0.024 |

Strain for a Shape Memory Polymer (“SMP”) would depend on delivery and final shape desired. If sent in a tube that is 2 mm wide (6 Fr.) and want to expand to 8 mm:

$$Strain = \frac{dl}{l} = \frac{6 mm}{2 mm} = 300 \% change$$

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A apparatus for closing a vascular puncture, comprising:
a first catheter having a proximal end, a distal end, a first balloon lumen, and a support balloon positioned at the distal end of the first catheter, the support balloon in pneumatic communication with the first balloon lumen;
a sheath having a distal end and a hood portion, the hood portion positioned at the distal end of the sheath;
a second catheter having a distal end, a second balloon lumen, and an expander balloon positioned at the distal end of the second catheter, the expander balloon in pneumatic communication with the second balloon lumen; and
a pusher assembly having a distal end, a pusher, and a bandage, the pusher assembly configured to be coaxial with, and located radially between, the first catheter and the sheath, and the pusher positioned at the distal end of the pusher assembly, and the bandage positioned at the distal end of the pusher.

2. The apparatus of claim 1, wherein a guide wire is located in the vasculature and the first catheter has a guide lumen configured such that the first catheter is advanced along the guide wire through the puncture into the vasculature.

3. The apparatus of claim 1, wherein the support balloon is configured to be inflated and/or deflated in situ by way of the first balloon lumen and the support balloon is made from a compliant material capable of providing support for the vasculature without deformation.

4. The apparatus of claim 1, wherein the first catheter has a bleed-back lumen which terminates at a bleed-back orifice, the bleed-back orifice positioned on the first catheter proximal to the support balloon.

5. The apparatus of claim 1, wherein the sheath is configured to be coaxial with, and in radially-spaced-apart relation to the first catheter.

6. The apparatus of claim 1, wherein the sheath is made from poly(urethane).

7. The apparatus of claim 4, wherein substantially all of the hood portion of the sheath is located proximal to the bleed-back orifice.

8. The apparatus of claim 1, wherein the hood portion is configured to expand from a first diameter to a second diameter, the second diameter being larger than the diameter of the puncture and larger than the diameter of the sheath in order to form an operable space over the puncture.

9. The apparatus of claim 8, wherein the expander balloon is configured to be inflated and/or deflated in situ by way of the second balloon lumen to expand the hood portion of the sheath.

10. The apparatus of claim 8, wherein the hood portion is made from a shape memory material.

11. The apparatus of claim 1, wherein the second catheter is configured to be coaxial with, and located radially between, the first catheter and the sheath.

12. The apparatus of claim 1, wherein the pusher has an expanded configuration and a collapsed configuration for insertion through an accessory lumen.

13. The apparatus of claim 12, wherein the pusher is configured to be biased to the expanded configuration such that when the pusher assembly is advanced through the sheath and reaches the hood portion, the pusher will expand to the expanded configuration.

14. The apparatus of claim 13, wherein the pusher comprises a plurality of push rods substantially parallel to one another when the pusher is in the collapsed configuration, and wherein the pushrods are configured to expand outwardly with respect to a central, longitudinal axis of the pusher.

15. The apparatus of claim 1, wherein the bandage is configured to be held in place on the puncture by the pusher.

16. The apparatus of claim 15, wherein the bandage has an orifice and the first catheter is configured to be positioned through the orifice such that the distal end of the first catheter remains inside the vasculature when the bandage is held on the puncture by the pusher.

17. The apparatus of claim 1 further comprising a lock for locking each component of the apparatus to each other to allow the proper longitudinal position of each component during use.

18. A method for closing a puncture of a blood vessel using an apparatus having a first catheter comprising a first balloon lumen and a support balloon in pneumatic communication with the first balloon lumen, a sheath having a hood portion, a second catheter comprising a second balloon lumen and an expander balloon in pneumatic communication with the second balloon lumen, and a pusher assembly having a bandage, the method comprising the steps of:
inserting the first catheter into the vessel through the puncture;
positioning the sheath outside of the puncture;
inflating the support balloon using the first balloon lumen until the balloon is substantially the same diameter as the vasculature;
inserting the second catheter into the sheath, such that the expander balloon is positioned in the hood portion of the sheath;
inflating the expander balloon using the second balloon lumen to expand the hood portion of the sheath;
deflating the expander balloon using the second balloon lumen;
positioning the pusher assembly within the hood portion of the sheath;
affixing the bandage, using the pusher assembly, on the puncture to close the puncture;
deflating the support balloon using the first balloon lumen; and
removing the first catheter from the vessel.

19. The method of claim 18, wherein the first catheter further comprises a bleed-back orifice and a bleed-back indicator, the bleed-back orifice positioned on the first catheter proximal to the support balloon, and the method further comprises the steps of:
advancing the first catheter within the vasculature until the bleed-back indicator shows that the bleed-back orifice is within the vessel; and
withdrawing the apparatus until the bleed-back indicator shows that the bleed-back orifice is no longer within the vessel.

20. A method for minimally-invasive closure of a puncture in a blood vessel, the method comprising the steps of:
disposing a support balloon in the blood vessel at the location of the puncture;
inflating the support balloon;
deploying a hood over the puncture;
pushing a bandage onto the blood vessel at the puncture and within the hood; and
removing the support balloon and hood.
